(19) United States
(12) Patent Application Publication
(10) Pub. No.: US 2005/0027012 A1
(43) Pub. Date: Feb. 3, 2005

(54) Tablets containing ambroxol

(75) Inventor: Anja Kohlrausch, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/888,362

(22) Filed: Jul. 9, 2004

(30) Foreign Application Priority Data
Jul. 16, 2003 (DE) DE 103 32 458
Dec. 20, 2003 (DE) DE 103 60 086

(51) Int. Cl. A61K 31/137; A61K 9/20

(52) U.S. Cl. 514/649; 424/464

(57) ABSTRACT

A tablet comprising a core portion comprising 150 mg to 1200 mg of ambroxol and a film coating surrounding the core portion.

### RELATED APPLICATIONS

This application claims priority to German patent application No. DE 103 32 458.5, filed Jul. 16, 2003, and German patent application No. DE 103 60 086.8, filed Dec. 20, 2003, each of which is hereby incorporated by reference in its entirety.

### FIELD OF THE INVENTION

The present invention relates to tablets containing the active substance ambroxol or one of the pharmacologically acceptable salts thereof, the ambroxol content of each tablet being in the range from 250 mg to 1000 mg.

### BACKGROUND OF THE INVENTION

Ambroxol (trans-4-(2-amino-3,5-dibromobenzylamino)cyclohexanol) is used as an expectorant in the form of syrups, elixirs, and tablets, and also as a local anesthetic in the form of a tablet for sucking. In addition, ambroxol displays good effects in the treatment of chronic pain, particularly in a daily dose of 500 mg per day or more.

Ambroxol-containing tablets which contain up to 75 mg of ambroxol per tablet are known in the art. As a high-dose formulation, ambroxol is administered in the form of a 1000 mg/mL injectable solution for the treatment of Respiratory Distress Syndrome (RDS) and for prenatal lung maturation.

For treating diseases, for example, chronic pain, which require a daily dose of ambroxol of 500 mg/day or more, a patient would be taking at least six of the 75 mg tablets which have hitherto been available. To give the patient an acceptable medication plan, several tablets would have to be replaced by a higher single dose per tablet.

The production of a higher-dose tablet is problematic. Thus, for example, care must be taken with the size of the tablet so as to avoid the rejection by the patient of a tablet which is too big.

Furthermore, it is difficult to manufacture a tablet with a high content of active substance which will also retain a short release time for the active substance as well as sufficient mechanical stability, while having good tablet-making qualities in the tablet press.

The aim of the present invention is therefore to produce a tablet having an ambroxol content of at least 150 mg, which has a short release time, sufficient mechanical stability, and good tabletting qualities.

### DESCRIPTION OF THE INVENTION

Surprisingly the aim outlined above can be achieved by means of the formulation described below.

The invention relates to a tablet containing a core and a film coating surrounding this core, characterized in that the core contains an ambroxol content of 150 mg to 1200 mg of ambroxol.

A tablet is preferred wherein the core contains an ambroxol content of 500 mg to 1000 mg, preferably 750 mg to 800 mg of ambroxol.

Also preferred is a tablet wherein the core contains one or more fillers selected from among pregelatinized starch, microcrystalline cellulose, hydroxypropylcellulose, cellulose, mannitol, erythritol, lactose, saccharose, calcium hydrogen phosphate, calcium carbonate, maize starch, sorbitol, and xylitol, preferably pregelatinized starch, microcrystalline cellulose, low-substituted hydroxypropylcellulose, mannitol, erythritol, and lactose, and most preferably pregelatinized starch, microcrystalline cellulose, and low-substituted hydroxypropylcellulose.

Particularly preferred is a tablet wherein the core contains one or more disintegration promoters selected from among croscarmellose sodium (cellulose carboxymethylether sodium salt, cross-linked), sodium starch glycolate, cross-linked polyvinylpyrrolidone (crospovidone), maize starch, microcrystalline cellulose, pregelatinized starch, and low-substituted hydroxypropylcellulose, preferably crospovidone, croscarmellose sodium, and sodium starch glycolate, and most preferably croscarmellose sodium and crospovidone.

Also particularly preferred is a tablet wherein the core contains one or more binders selected from among polyvinylpyrrolidone (povidone), copolymers of vinylpyrrolidone with other vinyl derivatives (copovidone), hydroxypropylmethylcellulose, methylcellulose, hydroxypropylcellulose, low-substituted hydroxypropylcellulose, and starch, preferably povidone, hydroxypropylmethylcellulose, and copovidone, most preferably povidone and copovidone.

Particularly preferred is a tablet wherein the core contains as lubricant magnesium stearate and/or sodium stearylfumarate, preferably magnesium stearate.

Of particular importance is a tablet wherein the film coating contains excipients selected from among talc, titanium dioxide, hydroxypropylmethylcellulose, and polyoxyethylene glycol. The film coating may optionally contain one or more synthetic or natural, pharmaceutically acceptable colorings, preferably iron oxide.

Also of particular significance is a tablet in which the proportion by weight of ambroxol in relation to the total mass of the core is 30-90 percent by weight (wt. %), preferably 40-90 wt. %, most preferably 60-70 wt. %.

Also of particular importance is a tablet wherein the proportion by weight of the film in relation to the total mass of the tablet is 2 to 4 wt. %, preferably 2 to 3 wt. %.

The invention further relates to a process for preparing the tablet according to the invention, in which the following process steps (a) to (f) are carried out in the sequence specified:
(a) mixing ambroxol or one of the pharmacologically acceptable salts thereof with pharmacologically acceptable excipients, optionally in the presence of a diluent, for example, microcrystalline cellulose;
(b) granulating the resulting mixture with a binder solution, for example, a solution of polyvinylpyrrolidone in water;
(c) drying the granules, e.g., in a fluidized bed dryer, followed by a screening step;
(d) mixing the granules obtained after the addition of further excipients, for example, a disintegration promoter (e.g., crospovidone), a binder (e.g., microcrystalline cellulose), and a lubricant, e.g., magnesium stearate);
(e) compressing the resulting mixture with a suitable tablet press; and
(f) coating the tablet core with a film.

The invention further relates to the use of the tablet according to the invention for preparing a pharmaceutical composition for the treatment of chronic pain, preferably chronic neuropathic pain or chronic nociceptive pain, most preferably chronic neuropathic pain.

The invention further relates to the use of the tablet according to the invention for preparing a pharmaceutical composition for the treatment of tinnitus.

The invention further relates to the use of the tablet according to the invention for preparing a pharmaceutical composition for the treatment of acute pain, preferably operative pain, toothache, pain caused by trauma, pain caused by burns, pain after stroke or myocardial infarct, pain caused by cramps, or pain caused by colic.

The invention further relates to the use of the tablet according to the invention for preparing a pharmaceutical composition for the treatment of epilepsy.

The proportion of filler in relation to the total core of the tablet according to the invention is kept within the range from 1 to 70 wt. %, preferably in the range from 5 to 50 wt. %, most preferably in the range from 20 to 30 wt. %.

The proportion of binder in relation to the total core of the tablet according to the invention is kept within the range from 1 to 20 wt. %, preferably in the range from 2 to 10 wt. %, most preferably in the range from 4 to 6 wt. %.

The proportion of disintegration promoter in relation to the total core of the tablet according to the invention is kept within the range from 1 to 20 wt. %, preferably in the range from 2 to 10 wt. %, most preferably in the range from 3 to 5 wt. %.

The proportion of lubricant in relation to the total core of the tablet according to the invention is kept within the range from 0.25 to 6 wt. %, preferably from 0.4 to 4 wt. %, most preferably from 0.5 to 2 wt. %.

The name ambroxol within the scope of the present invention denotes both the base ambroxol, and also the solvates or hydrates thereof. Where the content of ambroxol is given in mg or wt. % these are based on the ambroxol base.

Acids suitable for forming salts of ambroxol are for example hydrochloric acid, hydrobromic acid, sulfuric acid, phosphoric acid, nitric acid, oxalic acid, malonic acid, fumaric acid, maleic acid, tartaric acid, citric acid, ascorbic acid, and methanesulfonic acid, preferably hydrochloric acid.

The following procedure may be used, for example, to prepare the film-coated tablet according to the invention. Ambroxol or one of the pharmacologically acceptable salts thereof is premixed with a binder and optionally other excipients as mentioned above. The active substance premix thus obtained is then granulated in a fluidized bed granulator with an aqueous binder solution. Alternative methods of granulation with aqueous binder solutions are wet granulation in the intensive mixer or one-pot granulator or wet extrusion followed by screening, drying and dry screening of the granules.

The dried granules are screened, preferably with a 0.8 mm Comil screen. Other excipients such as disintegration promoters are added to the granulated active substance and then mixed in a gravity mixer, for example. Once the mixing process has ended, the mixture of active substance and excipient thus obtained is then compressed in a suitable tablet press to form the film-coated tablet cores according to the invention with the desired target weight and appropriate shape, size and shatter resistance.

In order to produce the film coating suspension both the essential and optional ingredients of the film coatings are taken up in a suitable solvent. According to the invention, water is preferably used as the solvent. When water is used as the solvent, the ingredients of the film coatings are partly in dispersed form. Once the coating suspension has been prepared, the tablet cores obtained previously are coated with the desired film in a suitable coating apparatus analogously to coating methods known in the art.

The Examples which follow illustrate the present invention without restricting its scope.

### EXAMPLES OF FORMULATIONS

### Example 1

### Film-Coated Ambroxol Tablet (750 mg)

| | mg/tablet | % per tablet | % of film |
|---|---|---|---|
| ambroxol HCl¹⁾ | 822.320 | 63.255 | |
| microcrystalline cellulose | 360.680 | 27.745 | |
| povidone | 65.000 | 5.000 | |
| croscarmellose sodium | 39.000 | 3.000 | |
| magnesium stearate | 13.000 | 1.000 | |
| purified water²⁾ | q.s. | | |
| Mass of tablet core | 1300.000 | 100.000 | |
| hydroxypropylmethylcellulose | 20.000 | 1.538 | 50.000 |
| polyethyleneglycol | 2.000 | 0.154 | 5.000 |
| titanium dioxide | 10.000 | 0.769 | 25.000 |
| talc | 7.200 | 0.554 | 18.000 |
| iron oxide red | 0.800 | 0.062 | 2.000 |
| purified water²⁾ | q.s. | | |
| Mass of film-coated tablet | 1340.000 | 103.077 | 100.000 |

¹⁾Corresponding to 750 mg of ambroxol base
²⁾no water left in the end product

### Example 2

### Film-Coated Ambroxol Tablet (750 mg)

| | mg/tablet | % per tablet | % of film |
|---|---|---|---|
| ambroxol HCl¹⁾ | 822.320 | 63.255 | |
| microcrystalline cellulose | 230.680 | 17.745 | |
| hydroxypropylcellulose, low | 130.000 | 10.000 | |
| subst. | | | |
| povidone | 65.000 | 5.000 | |
| crospovidone | 39.000 | 3.000 | |
| magnesium stearate | 13.000 | 1.000 | |
| purified water²⁾ | q.s. | | |
| Mass of tablet core | 1300.000 | 100.000 | |
| hydroxypropylmethylcellulose | 20.000 | 1.538 | 50.000 |
| polyethyleneglycol | 2.000 | 0.154 | 5.000 |
| titanium dioxide | 10.000 | 0.769 | 25.000 |
| talc | 7.200 | 0.554 | 18.000 |
| iron oxide red | 0.800 | 0.062 | 2.000 |
| purified water²⁾ | q.s. | | |
| Mass of film-coated tablet | 1340.000 | 103.077 | 100.000 |

¹⁾Corresponding to 750 mg of ambroxol base
²⁾no water left in the end product

### Example 3

### Film-Coated Ambroxol Tablet (500 mg)

| | mg/tablet | % per tablet | % of film |
|---|---|---|---|
| ambroxol HCl¹⁾ | 548.214 | 54.821 | |
| microcrystalline cellulose | 261.786 | 26.179 | |
| hydroxypropylcellulose, low | 100.000 | 10.000 | |
| subst. | | | |
| copovidone VA 64 | 50.000 | 5.000 | |
| crospovidone | 30.000 | 3.000 | |
| magnesium stearate | 10.000 | 1.000 | |
| purified water²⁾ | q.s. | | |
| Mass of tablet core | 1000.000 | 100.000 | |
| hydroxypropylmethylcellulose | 15.000 | 1.500 | 50.000 |
| polyethyleneglycol | 1.500 | 0.150 | 5.000 |
| titanium dioxide | 7.500 | 0.750 | 25.000 |
| talc | 5.400 | 0.540 | 18.000 |
| iron oxide red | 0.600 | 0.060 | 2.000 |
| purified water²⁾ | q.s. | | |
| Mass of film-coated tablet | 1030.000 | 103.000 | 100.000 |

¹⁾Corresponding to 500 mg of ambroxol base
²⁾no water left in the end product

### Example 4

### Film-Coated Ambroxol Tablet (1000 mg)

| | mg/tablet | % per tablet | % of film |
|---|---|---|---|
| ambroxol HCl¹⁾ | 1096.427 | 84.341 | |
| microcrystalline cellulose | 86.573 | 6.659 | |
| povidone | 65.000 | 5.000 | |
| croscarmellose sodium | 39.000 | 3.000 | |
| magnesium stearate | 13.000 | 1.000 | |
| purified water²⁾ | q.s. | | |
| Mass of tablet core | 1300.000 | 100.000 | |
| hydroxypropylmethylcellulose | 20.000 | 1.538 | 50.000 |
| polyethyleneglycol | 2.000 | 0.154 | 5.000 |
| titanium dioxide | 10.000 | 0.769 | 25.000 |
| talc | 7.200 | 0.554 | 18.000 |
| iron oxide red | 0.800 | 0.062 | 2.000 |
| purified water²⁾ | q.s. | | |
| Mass of film-coated tablet | 1340.000 | 103.077 | 100.000 |

¹⁾Corresponding to 500 mg of ambroxol base
²⁾no water left in the end product

1. A tablet comprising a core portion comprising 150 mg to 1200 mg of ambroxol and a film coating surrounding the core portion.

2. The tablet according to claim 1, wherein the core portion comprises 500 mg to 1000 mg of ambroxol.

3. The tablet according to claim 1, wherein the core portion further comprises pregelatinized starch, microcrystalline cellulose, hydroxypropylcellulose, cellulose, mannitol, erythritol, lactose, saccharose, calcium hydrogen phosphate, calcium carbonate, maize starch, sorbitol, or xylitol, or a mixture thereof.

4. The tablet according to claim 2, wherein the core portion further comprises pregelatinized starch, microcrystalline cellulose, hydroxypropylcellulose, cellulose, mannitol, erythritol, lactose, saccharose, calcium hydrogen phosphate, calcium carbonate, maize starch, sorbitol, or xylitol, or a mixture thereof.

5. The tablet according to claim 1, wherein the core portion further comprises croscarmellose sodium, sodium starch glycolate, crospovidone, maize starch, microcrystalline cellulose, pregelatinized starch, or low-substituted hydroxypropylcellulose, or a mixture thereof.

6. The tablet according to claim 2, wherein the core portion further comprises croscarmellose sodium, sodium starch glycolate, crospovidone, maize starch, microcrystalline cellulose, pregelatinized starch, or low-substituted hydroxypropylcellulose, or a mixture thereof.

7. The tablet according to claim 3, wherein the core portion further comprises croscarmellose sodium, sodium starch glycolate, crospovidone, maize starch, microcrystalline cellulose, pregelatinized starch, or low-substituted hydroxypropylcellulose, or a mixture thereof.

8. The tablet according to claim 4, wherein the core portion further comprises croscarmellose sodium, sodium starch glycolate, crospovidone, maize starch, microcrystalline cellulose, pregelatinized starch, or low-substituted hydroxypropylcellulose, or a mixture thereof.

9. The tablet according to claim 1, wherein the core portion further comprises povidone, copovidone, hydroxypropylmethylcellulose, methylcellulose, hydroxypropylcellulose, low-substituted hydroxypropylcellulose, or starch, or a mixture thereof.

10. The tablet according to claim 2, wherein the core portion further comprises povidone, copovidone, hydroxypropylmethylcellulose, methylcellulose, hydroxypropylcellulose, low-substituted hydroxypropylcellulose, or starch, or a mixture thereof.

11. The tablet according to claim 3, wherein the core portion further comprises povidone, copovidone, hydroxypropylmethylcellulose, methylcellulose, low-substituted hydroxypropylcellulose, or starch, or a mixture thereof.

12. The tablet according to claim 4, wherein the core portion further comprises povidone, copovidone, hydroxypropylmethylcellulose, methylcellulose, low-substituted hydroxypropylcellulose, or starch, or a mixture thereof.

13. The tablet according to claim 5, wherein the core portion further comprises povidone, copovidone, hydroxypropylmethylcellulose, methylcellulose, or starch, or a mixture thereof.

14. The tablet according to claim 6, wherein the core portion further comprises povidone, copovidone, hydroxypropylmethylcellulose, methylcellulose, or starch, or a mixture thereof.

15. The tablet according to claim 7, wherein the core portion further comprises povidone, copovidone, hydroxypropylmethylcellulose, methylcellulose, hydroxypropylcellulose, or starch, or a mixture thereof.

16. The tablet according to claim 8, wherein the core portion further comprises povidone, copovidone, hydroxypropylmethylcellulose, methylcellulose, hydroxypropylcellulose, or starch, or a mixture thereof.

17. The tablet according to claim 1, wherein the core portion further comprises magnesium stearate and/or sodium stearylfumarate.

18. The tablet according to claim 2, wherein the core portion further comprises magnesium stearate and/or sodium stearylfumarate.

19. The tablet according to claim 3, wherein the core portion further comprises magnesium stearate and/or sodium stearylfumarate.

20. The tablet according to claim 4, wherein the core portion further comprises magnesium stearate and/or sodium stearylfumarate.

21. The tablet according to claim 5, wherein the core portion further comprises magnesium stearate and/or sodium stearylfumarate.

22. The tablet according to claim 6, wherein the core portion further comprises magnesium stearate and/or sodium stearylfumarate.

23. The tablet according to claim 7, wherein the core portion further comprises magnesium stearate and/or sodium stearylfumarate.

24. The tablet according to claim 8, wherein the core portion further comprises magnesium stearate and/or sodium stearylfumarate.

25. The tablet according to claim 1, wherein the film coating comprises talc, titanium dioxide, polyoxyethylene glycol, hydroxypropylmethylcellulose, or iron oxide, or a mixture thereof.

26. The tablet according to claim 2, wherein the film coating comprises talc, titanium dioxide, polyoxyethylene glycol, hydroxypropylmethylcellulose, or iron oxide, or a mixture thereof.

27. The tablet according to claim 3, wherein the film coating comprises talc, titanium dioxide, polyoxyethylene glycol, hydroxypropylmethylcellulose, or iron oxide, or a mixture thereof.

28. The tablet according to claim 4, wherein the film coating comprises talc, titanium dioxide, polyoxyethylene glycol, hydroxypropylmethylcellulose, or iron oxide, or a mixture thereof.

29. The tablet according to claim 1, wherein the proportion by weight of ambroxol in relation to the total mass of the core portion is 30-90 wt. %.

30. The tablet according to claim 1, wherein the proportion by weight of the film in relation to the total mass of the tablet is 2 to 4 wt. %.

31. The tablet according to claim 29, wherein the proportion by weight of the film in relation to the total mass of the tablet is 2 to 4 wt. %.

32. A process for producing a tablet, comprising:
(a) mixing ambroxol or a pharmacologically acceptable salt thereof with one or more pharmacologically acceptable excipients, optionally in the presence of a diluent;
(b) granulating the resulting mixture with a binder solution;
(c) drying the granules and then screening the dried granules;
(d) mixing the granules obtained after the addition of further excipients, optionally a disintegration promoter, a binder, and a lubricant;
(e) compressing the resulting mixture with a suitable tablet press; and
(f) coating the tablet core with a film.
